(19) United States
(12) Patent Application Publication
(10) Pub. No.: US 2022/0047825 A1
(43) Pub. Date: Feb. 17, 2022

(54) A FLOW INDICATOR FOR OXYGEN ADDITION TO REBREATHING SYSTEMS

(71) Applicant: MIROLA IP AB, Örebro (SE)

(72) Inventor: Ola TELBY, Karlstad (SE)

(73) Assignee: MIROLA IP AB, Örebro (SE)

(21) Appl. No.: 16/765,422

(22) PCT Filed: May 9, 2019
(86) PCT No.: PCT/EP2019/061947

§ 371 Date: May 19, 2020

(51) Int. Cl. A61M 16/00

(52) U.S. Cl. CPC A61M 16/0045; A61M 16/0003; A61M 2202/0208; A61M 2205/3334; A61M 2205/584

(57) ABSTRACT

The invention relates to a flow indicator preferably to be used in oxygen therapy treatment of patients where a clear flow or no flow state must be signaled for the supply of oxygen.

According to the inventive flow indicator are two cylindrical bodies used, one (30) movable and one (10) stationary, and with one extended flow restriction passage (80) that forces the movable cylindrical body away from a sight glass (40), instead exposing the stationary cylindrical body. The viewable cylindrical part of the movable part may be colored red (R), and the viewable cylindrical part of the stationary body may be colored green (G).

A flow indicator with quick and stable indication of developed flow is obtained.

### BACKGROUND OF THE INVENTION

The present invention relates to a flow indicator which provide either a first visual signal for developed oxygen flow or alternatively a second distinctive visual signal for non-developed flow to a rebreathing mask.

### PRIOR ART

Flow meters are used when the proper development of flow is important for critical functions, and for rebreathing systems to assure proper oxygen therapy to the patient. Rebreathing systems and accessories therefor are classified under A61M 16, Devices for influencing the respiratory system of patients by gas treatment . . . , in the Cooperative Patent Classification.

In hospital environment is most often the order of flow necessary to monitor for controlling proper concentration of oxygen in breathing systems, Typically, oxygen is supplied in excess and most of the oxygen given to patients is waisted, but of less interest as the supply of oxygen is almost limitless.

Rebreathing systems to be used at site of accidents are developed, and in those systems are often rebreathing bags used that collect exhaled volumes, after first passing a CO₂ filter. In such systems may oxygen be added in small amounts replacing the CO₂ caught in the filter, and hence could these rebreathing systems be equipped with a rather small oxygen bottle suitable for rescue vehicles. In such emergency situations should monitoring systems be kept simple and operable under any orientation of the system or parts therefor.

Conventional flow meters for breathing apparatus most often use a ball, or another shaped body, which rises up or fall down in a vertically oriented sight glass tube, and where the vertical position may be used as a measurement of the order of flow. When flow is interrupted will the ball settle down due to gravity. Examples of such flow meters may be seen in U.S. Pat. Nos. 3,196,673 or 2,655,041. These flow sensors may function properly in hospital environment where oxygen supply is obtained from stationary wall mounted outlet taps, or when using emergency carriages, with components such as flow meters fixed to the carriage or the wall mounted taps in a predetermined vertical position.

Other types of simple in-line flow meters has been suggested, such as flow meters with a scale on a transparent housing. Examples of flow meters with scale are shown in;
In GB 645490 (1950) is a flow meter disclosed having a transparent housing and a spring biased piston movable within the housing for indicating the rate of flow of fluid through the housing in accordance with the position of the piston relative to a scale on the housing.
In GB 2,073,893 (1981) is disclosed another flow meter similar to GB 645490.
In U.S. Pat. No. 4,389,901 (1983) is a fluid flow meter disclosed where an indicator piston is gradually opening a flow passage around a conical throttle body.
In US 2007/0221223 (2007) is a similar gas flow meter proposed. Here is a spring biased piston arranged in a viewable channel, and where the position of the piston indicate the flow rate. The problem with these flow meters is the difficulties making a proper reading of the true flow rate.

Another flow meter is disclosed in U.S. Pat. No. 4,986,133 (1991), where the flow sensor us equipped with easily changeable flow restriction discs, making it possible to alter measurement to different flow ranges.

Further, in U.S. Pat. No. 2,638,582 is disclosed a check valve with an external magnetic indicator. When pressure is applied on a movable pin is a magnetic body lifted by the pin against the action of a spring thus changing the position of the magnetic indicator.

Above examples of flow meters often includes a metering function of the flow rate, and to read off the rate of flow it is necessary to get close to flow meter and align eyes with the scale on the flow meter with the small indicator. However, in oxygen treatment equipment used on site of accidents, the need is to get a clear signal of flow or no flow to the patent, and such indication must be easily seen by rescue personnel from whatever position they may be in. This is especially important in accidents with a lot of patients needing oxygen therapy.

Small rebreathing systems has been developed which utilize the oxygen more economically, and hence need only small oxygen bottles for assisting oxygen enriched breathing to patients. These rescue kits are made so small such that a rescue vehicle may bring along 10 or more rescue kits. The size of the rescue kit is essential for this capacity and small flow meters or indicators need to be developed that are easy to manufacture, inexpensive and easy to read, as well as having a stable indication of flow or no flow status. An example of such small rebreathing system may be seen in WO2014/035330, disclosing a rebreathing system used for extending supply of oxygen to the rebreathing circuit.

### SUMMARY OF THE INVENTION

The invention solves these problems by using two cylinder shaped bodies in the flow meter, one stationary and one movable, where the external surface of these bodies may be seen in a flow housing with a sight glass. By applying a longer flow restriction passage between these cylindrical shaped bodies, could a calibrated flow meter with quick response to changes between flow or no flow conditions be obtained.

The invention relates to a flow indicator for indicating developed flow of oxygen into rebreathing systems comprising
an elongated flow indicator housing with an oxygen inlet in one end and an oxygen outlet in the other end of the elongated flow indicator housing; and
with at least a part of the elongated flow indicator housing made in a transparent material enabling visualization of at least a part of the flow channel trough said elongated flow indicator housing, and
a movable indicator body located inside of said elongated flow indicator housing; and
a bias member with one end fastened to a stationary anchor of the elongated flow indicator housing and the other end fastened to an anchor of the movable indicator body,
which bias member apply a force to the movable indicator body towards a closing seat preventing flow through the flow indicator housing, and
when oxygen pressure is applied to the oxygen inlet in said flow indicator housing is the movable indicator body pushed away from the closing seat allowing passage of oxygen flow.

The inventive flow indicator has an axially directed flow restriction passage arranged in a coaxial fashion between a stationary cylindrical part of the elongated flow indicator housing and a complementary cylindrical part of the movable indicator body, said flow restriction passage in one end connected to an inlet chamber in turn connected to the oxygen inlet and in the other end connected to an outlet chamber in turn connected to the oxygen outlet, and with the closing seat arranged between an axially facing surface of the stationary part of the elongated flow indicator housing and an axially facing surface of the movable indicator body. The arrangement of two coaxially oriented cylindrical parts with a flow restriction passage therebetween provides the basic principles enabling an indicator with fast response, indicating either no flow or developed flow. The bias member may preferably be a coil spring member as shown in attached figures, but any equivalent bias member may be used.

In a preferred embodiment of the inventive flow indicator has the part of the elongated flow indicator housing made in a transparent material an axial extension over the flow indicator housing from the first axial position of the closing seat, close to one of the inlet or outlet ends, to an axially distant second position closer to the other inlet or outlet end, with a distance between said first and second positions exceeding at least 50% of the axially length of the flow restriction passage. And more preferably is the distance between said first and second positions exceeding at least 75% of the axially length of the flow restriction passage.

In yet a preferred embodiment of the inventive flow indicator is the movable indicator body displaceable by the developed flow of oxygen to an axial position away from the part of the elongated flow indicator housing made in a transparent material, and with the outer part of the movable indicator body preferably being colored in a first signal color, signaling said first signal color through the transparent part of the housing when no flow of oxygen is developed, and when displaced from the part of the elongated flow indicator housing made in a transparent material when oxygen flow is developed instead expose a second signal color through the transparent part of the housing.

In yet a preferred detailed embodiment of the inventive flow indicator is the movable indicator body shaped as a cylinder closed in one end and in the other open end of the cylinder comprise the axially facing surface that forms the closing seat, and with an axial length of the cylinder exceeding that of the axial length of the part of the elongated flow indicator housing made in a transparent material, and which outer cylindric surface of the movable indicator body preferably colored in red.

In yet a further preferred detailed embodiment of the inventive flow indicator is a stationary inner cylinder located coaxially of the movable indicator body, which inner cylinder also has an axial length of the cylinder exceeding that of the axial length of the part of the elongated flow indicator housing made in a transparent material, and with the outer cylindric surface of the stationary inner cylinder preferably colored in green.

In a further detailed embodiment of the inventive flow indicator has the axially directed flow restriction passage has a progressively increasing flow area as the movable indicator body lifts from the closing seat. The progressively increasing gap of the flow restriction passage induce a higher pressure drop when the pressurized oxygen pass through the first narrower gap, thus establishing a higher pressure differential over the movable indicator body and hence a higher opening force. As the position of the movable indicator body expose successively larger gaps in the flow restriction passage is the pressure differential slightly decreased.

In another preferred embodiment of the inventive flow indicator is the axially directed flow restriction passage arranged in a coaxial fashion between a stationary cylindrical part of the elongated flow indicator housing and a complementary cylindrical part of the movable indicator body, and said flow passage show a step wise increase of flow area as the movable indicator body lifts from the closing seat, and wherein each step of flow increase is developed over an axial length of the axial flow restriction passage exceeding 10%, preferably 20%, of the total length of the axial flow restriction passage. By using two cylindrical parts for establishment of the flow restriction passage, could the step wise increase of the gap of the flow restriction passage be machined to size easily between these coaxially arranged parts, using for example drills with successively increasing diameter or machined to size in a lathe. It should however be considered that the flow restriction passage may be developed between other cylindrical surfaces than the ones shown in figures, i.e. developed between an inner stationary cylindric surface and an outer movable cylindric indicator body, and one such alternative may develop the flow restriction passage between the outer surface of the movable cylindric indicator body and the inwardly directed cylindric surface of the flow indicator housing.

In a preferred embodiment of the inventive flow indicator is the part of the elongated flow indicator housing made in a transparent material running over the entire circumference of the elongated flow indicator housing, exposing the outer surface of the movable indicator body or alternatively the outer surface of the stationary inner cylinder, depending on developed flow of oxygen and hence the axial position of the movable indicator body. This means that the flow indicator may be viewed by rescue personnel irrespective of any rotational position of the flow meter.

Finally, in a detailed embodiment of the inventive flow meter is the axial length of the part of the elongated flow indicator housing made in a transparent material at least 10 mm long, with the axial length of the outer surface of the movable indicator body or alternatively the outer surface of the stationary inner cylinder as well as the flow restriction passage, all exceeding the axial length of the part of the elongated flow indicator housing made in a transparent material. A 10 mm long sight glass will provide a minimum but easily viewable indicator window, with signal members at least as long being able to provide alternatively a no flow or developed flow indication. Preferably is the axial length of the part of the elongated flow indicator housing made in a transparent material in the range 10-30 mm. There is a trade off between cost and clear indication, and an axial length of at least 10 mm will enable a short and inexpensive flow meter still having a clearly viewable indication.

### LIST OF DRAWINGS

In the following schematic drawings may same details not be numbered in additional figures.

FIG. 1; Show an assembled flow meter according to the invention in a cross section view;

FIG. 2; Show the stationary cylindrical insert as such;

FIG. 3; Show the movable cylindrical insert as such;

FIG. 4a; Show the stationary and movable cylindrical inserts in a no flow state;

FIG. 4b; Show the stationary and movable cylindrical inserts in a fully developed flow state;

FIG. 5a; Show a side view of the flow indicator in a no flow state;

FIG. 5b; Show a side view of the flow indicator in a fully developed flow state;

FIG. 6; Show an alternative embodiment of the flow restriction passage between the two cylindrical inserts.

### DETAILED DESCRIPTION OF THE INVENTION

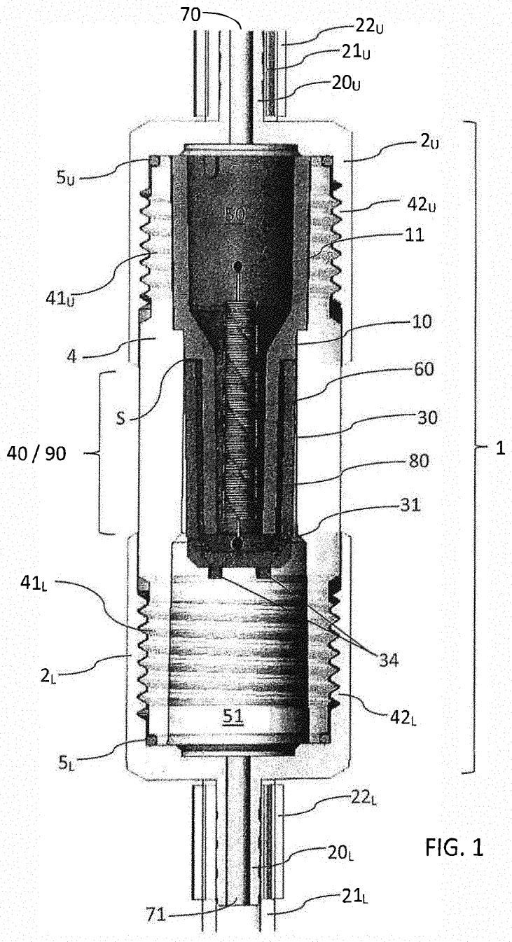

In FIG. 1 is shown the inventive flow indicator in a cross section view when installed in an oxygen supply hose 21.

The flow indicator housing 1 is in this embodiment manufactured and mounted in an oxygen supply hose 21 by assembly of only 10 different parts; i.e.
A single flow channel housing 4, preferably made in one piece of transparent material such as carbonate plastic;
Two end pieces, an upper and lower end piece, 2_{U} and 2_{L} respectively; Said end pieces attached to the flow channel housing by threads 42_{U}, 42_{L} engaging threads 41_{U}, 41_{L} on the flow channel housing. The end pieces integrated with an upper and lower hose nipple 20_{U} and 20_{L}.
Two standard O-ring seals 5_{U} and 5_{L} providing a pressure seal between the flow channel;
a stationary cylindrical part 10 of the elongated flow indicator housing, in this embodiment made as a separate part.
a movable indicator body 30; and
a bias member 60, and
finally, 2 hose clamps 22_{U} and 22_{L} providing a closing force on the hose towards the hose nipples.

The flow indicator hosing 1 has an oxygen inlet 70 through the hose nipple 20_{U} in the upper end, in turn connected to an inlet chamber 50, and an oxygen outlet 71 through the hose nipple 20_{L} in the lower end, in turn connected to an outlet chamber 51. The bias member 60, here a coil spring, is connected In one end to a stationary anchor 11 and in the other end to a movable anchor integrated with the movable indicator body. As long as the inlet chamber is not pressurized with oxygen is a sealing seat S developed between the stationary cylindric insert 10 and the movable indicator body 30, preventing flow through the flow indicator.

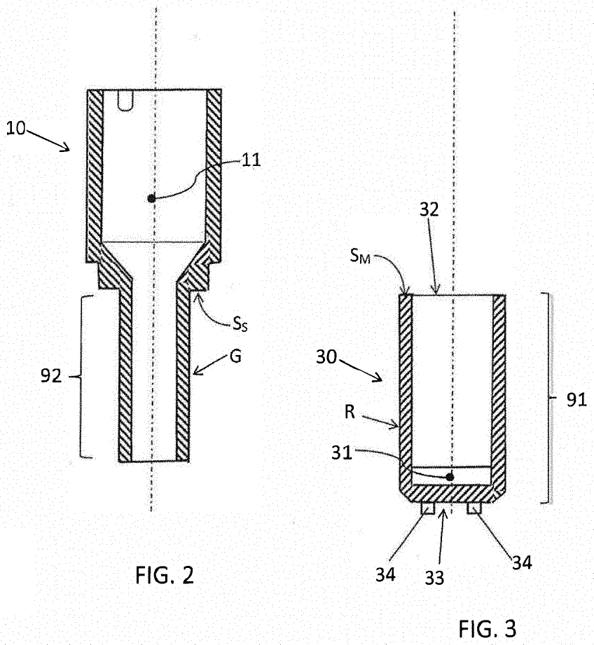

The stationary cylindrical part 10 of the elongated flow indicator housing, in this embodiment made as a separate part, is shown separately in FIG. 2. The essential cylindrical part that aligns with the transparent 40 part of the housing is the lower part 92. The outer surface 92 is preferably given a distinctive signal color, preferably green, as this entire surface is intended to be seen when flow of oxygen is developed. The part of the stationary cylindrical part 10 that form the sealing S is the axially facing shoulder S_{S}. An anchor 11 for the bias member, i.e. a coil spring end, is provided in form of a pin 11 running across the inlet chamber 50.

The movable indicator body 30 is shown separately in FIG. 3. The essential cylindrical part that aligns with the transparent part 40 of the housing is the outwardly facing surface 91. The outer surface 91 is preferably given a distinctive signal color, preferably red, as this surface is intended to be seen when no flow of oxygen is developed, and the movable indicator body settles with the circular end face S_{M} on the axially facing shoulder S_{S} of the stationary part. An anchor for the bias member, i.e. a coil spring end, is provided in form of a pin or a hook 31 mounted close to the closed gable end 33. The movable indicator body may also be provided with at least one smaller end projection 34 that prevents the closed gable end 33 to cover and close the oxygen outlet 71 from the outlet chamber 51.

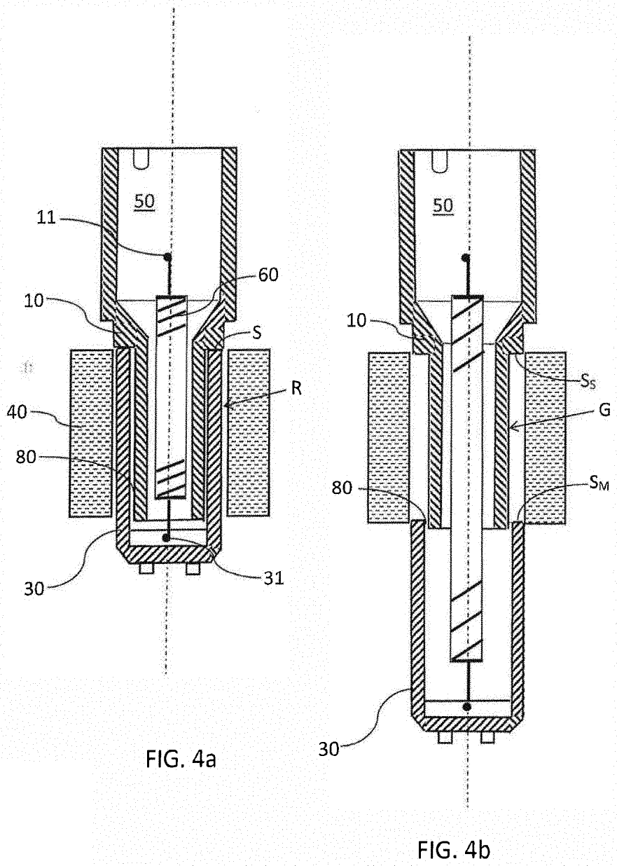

The interplay between the stationary cylindrical part 10 of the elongated flow indicator housing and the movable indicator body 30 is shown in FIGS. 4a and 4b, where FIG. 4a show the relative positions between these parts 10/30 as well as the transparent part of the flow indicator housing 40 in a no flow state, and FIG. 4b show the relative positions between these parts 10/30/40 in a fully developed flow state.

When no flow is developed, which may occur if the inlet chamber is not connected to a pressurized oxygen source or if the outlet channel 71 is blocked, the movable indicator body 30 will settled down on the sealing seat S by the action of the bias member 60. The outer surface R of the movable indicator body 30 is then seen through the transparent part of the flow indicator housing 40. In this starting position is a flow restriction passage 80 formed between the outer surface of the stationary cylindrical part 10 and the inner cylindrical surface of the movable indicator body 30.

When the inlet chamber 50 is connected to a pressurized oxygen source is the oxygen pressure at the inlet pressure applied onto the gable end 33 of the movable indicator body 30, and thus push the body 30 downward opening the passage through the sealing seat S. The flow of oxygen must pass the flow restriction passage 80 before passing the sealing seat S and is therefore subjected to pressure drop. The pressure in the outlet chamber 51, i.e. working on the other side of the gable end 33, will be lower than the pressure in the inlet chamber 50. The order of pressure drop established in the flow restriction passage is directly related to the gap in the passage 80. In FIG. 4b is shown the end position of the movable indicator body 30 when oxygen flow is established. This position will be kept as long as flow is developed, but as soon as flow is interrupted will the pressure on both sides of the closed gable end 33 assume same pressure, and the bias member will return the movable indicator body towards the position shown in FIG. 4a.

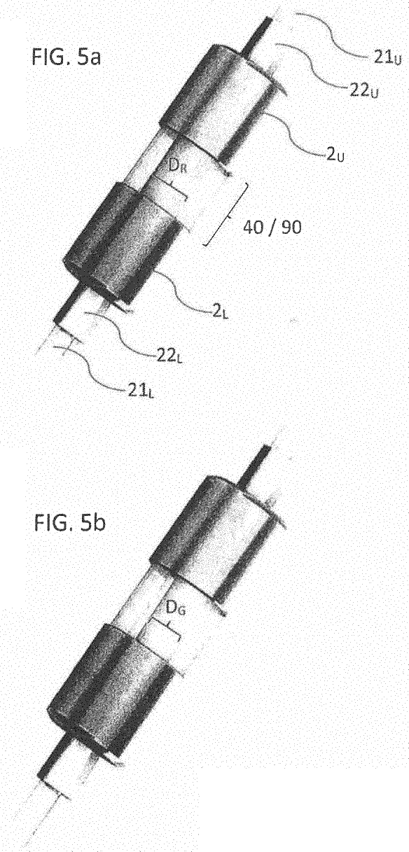

In FIG. 5a is shown a side view of the flow indicator in a no flow state, where the outer surface of the movable indicator body 30 Is viewable through the transparent part 40 of the flow indicator housing 1. In FIG. 5b is shown a side view of the flow indicator in a developed flow state, where the outer surface of the stationary cylindrical part 10 is viewable through the transparent part 40 of the flow indicator housing 1. As seen from previous figures is the diameter D_{R} (signaling “Red” in no flow state) slightly larger than the diameter D_{G} (signaling “Green” in a developed flow state).

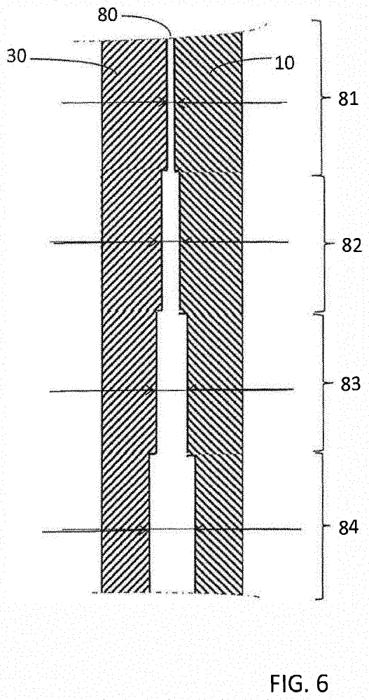

In FIG. 6 is an alternative configuration of the flow restriction passage 80 disclosed, which in contrast to previous embodiments do not have a constant gap in the flow restriction passage 80 over the entire stroke of the movable indicator body 30, but instead Is designed with a progressively increasing gap in the flow restriction passage 80. In this embodiment is the flow restriction passage designed with stepwise increase of gap in 4 stages and on both cylindrical surfaces of the movable indicator body 30 as well as the stationary cylindrical part 10. In this figure is the order of gap increase exaggerated and the step wise increase may be obtained from surface modification of only one surface and not both as shown here.

The cross section of the flow restriction passage 80 in the alternative with a constant gap size over the stroke length, is considerably smaller than the flow area through the hose 21 and the hose nipple and other flow sections in the flow indicator. Assuming that a hose 21 with a flow area of about 5 mm is used, then the flow restriction passage should have a total flow area of less than 50% of the hose, preferably less than 10-25% of the flow area of the hose. This narrow flow restriction passage may easily be machined to size in the two cylindrical parts, i.e. the movable indicator body 30 and the stationary cylindrical insert 10, or alternatively between the inside surface of the flow channel housing 4 and the outer surface of the movable indicator body 30.

Hence, in a total flow area in the flow restriction passage with a constant gap size may lie between 0.3-1.5 mm². When using a hose with a flow area of 3 mm². total flow area in the flow restriction passage with an increasing gap size, as shown in FIG. 6, may change gap size in steps between 0.3-1.5 mm² when using a hose with a flow area of 3 mm², wherein each step wise increase may lie in the order of 0.1-0.3 mm².

1. A flow indicator for indicating developed flow of oxygen into rebreathing systems comprising
an elongated flow indicator housing with an oxygen inlet in one end and an oxygen outlet in the other end of the elongated flow indicator housing and with at least a part of the elongated flow indicator housing made in a transparent material enabling visualization of at least a part of the flow channel through said elongated flow indicator housing, and
a movable indicator body located inside of said elongated flow indicator housing;
a bias member with one end fastened to a stationary anchor of the elongated flow indicator housing and the other end fastened to an anchor of the movable indicator body, which bias member apply a force to the movable indicator body towards a closing seat preventing flow through the flow indicator housing, and when oxygen pressure is applied to the oxygen inlet in said flow indicator housing is the movable indicator body pushed away from the closing seat allowing passage of oxygen flow, wherein an axially directed flow restriction passage is arranged in a coaxial fashion between a stationary cylindrical part of the elongated flow indicator housing and a complementary cylindrical part of the movable indicator body, said flow restriction passage in one end connected to an inlet chamber in turn connected to the oxygen inlet and in the other end connected to an outlet chamber in turn connected to the oxygen outlet, and with the closing seat arranged between an axially facing surface of the stationary part of the elongated flow indicator housing and an axially facing surface of the movable indicator body.

2. A flow indicator according to claim 1, wherein the part of the elongated flow indicator housing made in a transparent material has an axial extension over the flow indicator housing from the first axial position of the closing seat, close to one of the inlet or outlet ends, to an axially distant second position closer to the other inlet or outlet end, with a distance between said first and second positions exceeding at least 50% of the axially length of the flow restriction passage.

3. A flow indicator according to claim 2, wherein the distance between said first and second positions exceeds at least 75% of the axially length of the flow restriction passage.

4. A flow indicator according to claim 1, wherein the movable indicator body is displaceable by the developed flow of oxygen to an axial position away from the part of the elongated flow indicator housing made in a transparent material, and with the outer part of the movable indicator body preferably being colored in a first signal color, signaling said first signal color through the transparent part of the housing when no flow of oxygen is developed, and when displaced from the part of the elongated flow indicator housing made in a transparent material when oxygen flow is developed instead expose a second signal color through the transparent part of the housing.

5. A flow indicator according to claim 4, wherein the movable indicator body is shaped as a cylinder closed in one end and in the other open end of the cylinder comprise the axially facing surface that forms the closing seat, and with an axial length of the cylinder exceeding that of the axial length of the part of the elongated flow indicator housing made in a transparent material, and with the outer cylindric surface of the movable indicator body preferably colored in red.

6. A flow indicator according to claim 5, wherein a stationary inner cylinder is located coaxially of the movable indicator body, which inner cylinder also has an axial length of the cylinder exceeding that of the axial length of the part of the elongated flow indicator housing made in a transparent material, and with the outer cylindric surface of the stationary inner cylinder preferably colored in green.

7. A flow indicator according to claim 1, wherein the axially directed flow restriction passage has a progressively increasing flow area as the movable indicator body lifts from the closing seat.

8. A flow indicator according to claim 7, wherein the axially directed flow restriction passage is arranged in a coaxial fashion between a stationary cylindrical part of the elongated flow indicator housing and a complementary cylindrical part of the movable indicator body, and said flow passage shows a step wise increase of flow area as the movable indicator body lifts from the closing seat, and wherein each step of flow increase is developed over an axial length of the axial flow restriction passage exceeding 10%, preferably 20%, of the total length of the axial flow restriction passage.

9. A flow indicator according to claim 1, wherein the part of the elongated flow indicator housing made in a transparent material runs over the entire circumference of the elongated flow indicator housing, exposing the outer surface of the movable indicator body or alternatively the outer surface of the stationary inner cylinder, depending on developed flow of oxygen and hence the axial position of the movable indicator body.

10. A flow indicator according to claim 1, wherein the axial length of the part of the elongated flow indicator housing made in a transparent material is at least 10 mm long, with the axial length of the outer surface of the movable indicator body and the outer surface of the stationary inner cylinder as well as the flow restriction passage, all exceeding the axial length of the part of the elongated flow indicator housing made in a transparent material.
